(19) United States
(12) Patent Application Publication
(10) Pub. No.: US 2018/0206734 A1
(43) Pub. Date: Jul. 26, 2018

(54) WRIST TYPE APPARATUS FOR MEASUREMENT OF CARDIOVASCULAR HEALTH, SYSTEM, AND METHOD THEREOF

(71) Applicant: KENTEC INC., TAOYUAN COUNTY (TW)

(72) Inventors: CHIA-CHUN LIN, TAICHUNG CITY (TW); KUO-CHING CHIANG, TAOYUAN CITY (TW)

(21) Appl. No.: 15/411,929

(22) Filed: Jan. 20, 2017

(51) Int. Cl. A61B 5/0205; A61B 5/00; A61B 5/04

(52) U.S. Cl. CPC A61B 5/0205; A61B 5/681; A61B 5/04087; A61B 5/04012; A61B 2560/0223; A61B 5/7278

(57) ABSTRACT

The disclosure is related to a wrist type apparatus, and a system for measurement of cardiovascular health, and a method for the same. The wrist type apparatus is preferably a wrist type blood pressure device that includes an electrode-based module and a piezoelectric sensor module. The electrode-based module has three electrodes that are used to contact the skin of a human for generating a first set of data by measuring a target heartbeat. The piezoelectric sensor module is used to generate a second set of data by measuring a pulse wave produced by the target heartbeat corresponding to the first set of data. When the two sets of data are received by a processor, it is able to calculate a cardiovascular health value in accordance with cardiovascular health marker(s) that are selected from arterial stiffness, blood pressure, heart rate, pulse transit time, and pulse wave velocity.

### BACKGROUND

### 1. Technical Field

The present disclosure is related to an apparatus, a system, and a method for measurement of cardiovascular health in the field of diagnosis, and more particularly to the apparatus, system and method for identifying cardiovascular health by monitoring both the electrocardiographic source and piezoelectric pulse source.

### 2. Description of Related Art

Cardiovascular disease is the leading threat to human health in the world and also a major global health problem. It encompasses a wide gamut of disorders involving the heart and blood vessels that are typically linked. In the developed world, cardiovascular conditions such as stroke and myocardial infarction are leading killers directly caused by atherosclerosis, arterial stiffness, and hypertension.

As with any disease, prevention is far and away the better choice than post-crisis treatment, whenever it is possible. Annually, billions of dollars around the world could be saved in healthcare costs if cardiovascular conditions are well managed before they become life-threatening. Standards of living, particularly for the elderly, would also be dramatically improved if the cardiovascular conditions are caught and prevented at an early stage. To prevent cardiovascular diseases, blood pressure (BP) is a key issue for the traditional diagnosis and monitoring methods.

Conventional non-invasive blood pressure monitoring methods include those involving a cuff sphygmomanometer that has an inflatable cuff used to restrict blood flow, e.g., over the arm, and a measuring unit used to determine if the pressure blood flow starts and is unimpeded. However, the conventional cuff sphygmomanometer may be uncomfortable as it is worn on the arm; further, the cuff sphygmomanometer may generate data pertaining only to the position and time that it is worn and used. Therefore, the conventional cuff sphygmomanometer is unable to bear significantly accurate medical diagnoses. The conventional cuff sphygmomanometer can also be inaccurate for various reasons, such as emotional state of the user at the time of measurement, measurement times per day, and misjudgment on the user's part.

Invasive means for blood pressure monitoring, typically such as an intra-arterial catheter, may derive more accurate data and be able to achieve constant monitoring of blood pressure. However, the invasive means may run serious risks of arterial injury and infection.

Many modern devices and techniques for monitoring cardiovascular health in the prior art focus on fixed BP values. Also, the blood pressure in and of itself fails to provide a complete measurement of a particular individual's health since the values of blood pressure and health effects vary greatly between individuals. It is also difficult to achieve the precise fixed systolic/diastolic values by means of BP measuring devices known in the art. These devices tend to provide estimates only, and are non-continuous, momentary spot measurements that are subject to the effects of the individual's emotional and environmental circumstances at the time the measurement is taken.

Algorithms for estimating blood pressure using pulse transit time (PTT) are known in the art. Many doctors would agree that measurement of the arterial stiffness and the elasticity of an individual's arterial walls would be a more accurate indicator than a fixed BP value for any particular individual's cardiovascular health.

Arterial stiffness has previously been measured using both invasive and non-invasive methods. The non-invasive methods tend to fit into three types: 1) measuring Pulse Wave Velocity (PWV), such as with Doppler ultrasound, applanation tonometry, or MRI; 2) relating change in diameter (or area) of an artery to distending pressure, such as with ultrasound or MRI; or 3) assessing arterial pressure waveforms, such as with applanation tonometry.

Both pulse transit time (PTT) and pulse wave velocity (PWV) have been suggested for assessment of arterial stiffness.

Health monitoring via smailphones and other mobile devices is a burgeoning industry, with commercially available apps and hardware has also produced a smailphone ECG which incorporates electrodes into a wireless case that snaps onto the back of a smailphone. Bluetooth® heart rate detecting chest straps that communicate with smailphones are known in the art as well, and are also commercially available. Numerous devices or systems that utilize ECG and PPG to measure BP, PTT or other cardiovascular indicators are also known in the art.

### SUMMARY

In the disclosure of the present disclosure, a wrist type apparatus for measurement of cardiovascular health, a system, and a method for the same are provided. The system constantly monitors cardiovascular health using an electrocardiography (ECG) source synchronized with a piezoelectric pulse source without requiring invasive techniques, ongoing expensive or large-scale external scanning procedures. The components of the apparatus, structure and a calibration method of blood pressure in accordance with the present disclosure can collect more accuracy synchronized measurements on a continuous basis over an extended period of time so as to determine trending of an individual's personal cardiovascular markers over time. The monitoring over time allows for sustained biometric measurements, leading to clarification of an individual user's biometric signature, from which abnormalities in the rate of circulatory degeneration can be determined, thus allowing for the application of preventive measures before a health crisis occurs. The disclosure also provides a method using a mobile device to monitor, either continuously or intermittently, the marker selected from arterial stiffness, blood pressure, heart rate, pulse transit time, and pulse wave velocity.

A wrist type apparatus which comprises a main portion connected with a cuff portion, exemplarily the wrist type blood pressure device, for measurement of cardiovascular health is provided in the present disclosure. The wrist type apparatus includes a display unit disposed on the main portion; an electrode-based module, being an electrocardiographic source and having at least three electrocardiography (ECG) electrodes wherein each ECG electrode connects to a corresponding contact window that is used to contact skin, for generating a first set of data by measuring a target heartbeat; a piezoelectric sensor module, being a piezoelectric pulse source, having at least one piezoelectric pulse sensor wherein each piezoelectric pulse sensor connects to a corresponding contact window that is used to contact skin, for generating a second set of data by measuring a pulse wave produced by the target heartbeat corresponding to the first set of data; and a processor, electrically connected with the display unit, the electrode-based module and the piezoelectric sensor module, and in communication with both the electrode-based module and the piezoelectric sensor module for receiving the first set of data and the second set of data and accordingly calculating at least one cardiovascular health value being shown on the display unit. The at least three ECG electrodes has a first ECG electrode, a second ECG electrode and a third ECG electrode, in which the first ECG electrode is disposed on a same side of the display unit; the second ECG electrode, the third ECG electrode and one piezoelectric pulse sensor are disposed on a skin side of the cuff portion.

In the method for measurement of cardiovascular health by monitoring at least one cardiovascular health marker over time, adapted to the wrist type apparatus mentioned above, an ECG signal for the target heartbeat using an electrode-based module of the wrist type apparatus is obtained and is used to generate the first set of data when a first area of a user's body is contacted with the corresponding contact windows of the second ECG electrode and the third ECG electrode, and a second area of the user's body touches the corresponding contact window of the first ECG electrode; after that, a piezoelectric pulse signal for a pulse wave from the target heartbeat using a piezoelectric sensor module is also obtained and is used to generate a second set of data when the first area of the user's body is contacted with the corresponding contact window of the first piezoelectric pulse sensor. The first set of data and the second set of data are transmitted to the processor for calculating the at least one cardiovascular health value in accordance with the at least one cardiovascular health marker using the first and second sets of data.

It is noted that the cardiovascular health marker is selected from arterial stiffness, blood pressure, heart rate, pulse transit time, and pulse wave velocity. The electrode-based module includes at least three ECG electrodes with corresponding contact windows, and the piezoelectric sensor module includes at least one piezoelectric pulse sensor with corresponding contact window. The contact windows are all disposed over a surface of the wrist type apparatus, and the ECG signal and the piezoelectric pulse signal are obtained through the corresponding contact windows of the at least three ECG electrodes and the at least one piezoelectric pulse sensor to contact the skin of a human body. Furthermore, the corresponding contact window of the at least one piezoelectric pulse sensor can be encapsulated by room temperature vulcanization (RTV) silicone rubber for a better signal receiving effectiveness.

In one aspect of the present disclosure, a system for measurement of cardiovascular health is provided. The system includes one or more wrist type apparatuses, and a cloud server connected with the one or more wrist type apparatuses over a network. The cloud server is used to collect the cardiovascular health value from every wrist type apparatus.

In order to further understand the techniques, means and effects of the present disclosure, the following detailed descriptions and appended drawings are hereby referred to, such that, and through which, the purposes, features and aspects of the present disclosure can be thoroughly and concretely appreciated; however, the appended drawings are merely provided for reference and illustration, without any intention to be used for limiting the present disclosure.

### BRIEF DESCRIPTION OF THE DRAWINGS

FIG. 1 shows a circumstance of a user wearing a wrist type apparatus on the wrist in accordance with the present disclosure;

FIG. 2A, FIG. 2B, FIG. 2C, FIG. 2D and FIG. 2E show schematic diagrams respectively describing the structure and the sensor array of the wrist type apparatus in one embodiment of the present disclosure;

FIG. 3 shows a block diagram describing the wrist type apparatus in one embodiment of the present disclosure;

FIG. 4 shows a diagram depicting the pulse signals for the ECG signals and the piezoelectric pulses in a target heartbeat cycle; and

FIG. 5 shows a flow chart describing the method of measurement for cardiovascular health according to one embodiment of the present disclosure.

### DESCRIPTION OF THE EXEMPLARY EMBODIMENTS

Reference will now be made in detail to the exemplary embodiments of the present disclosure, examples of which are illustrated in the accompanying drawings. Wherever possible, the same reference numbers are used in the drawings and the description to refer to the same or like parts.

Disclosure herein is related to a device for measurement of cardiovascular health, a system and a method for the same. The device is preferably a wrist type apparatus to conduct the measurement of cardiovascular health. The device is directed to a wrist type apparatus, and applied to a system for measurement of cardiovascular health for identifying the cardiovascular health by monitoring both an electrocardiographic source and piezoelectric pulse source.

For the present disclosure, acronyms and definitions common in the fields of cardiovascular health and mechanical engineering are to apply. Specific terms include the following:

Electrocardiography (ECG) denotes the measurement of the electrical activity of the heart. An electrocardiogram is the record of the electrical activity of the heart while the signals are detected by electrodes attached to or contacting the skin.

Pulse Wave Velocity (PWV) denotes the velocity at which a pulse wave travels through the arterial tree.

Pulse Transit Time (PTT or T) denotes a time period it takes for a pulse wave to travel between two sites in the arterial tree.

Blood Pressure (BP or P) denotes the pressure exerted by circulating blood upon the walls of blood vessels. In every heartbeat, the blood pressure ranges between a maximum systolic pressure when the heart contracts, and a minimum diastolic pressure when the heart is at rest.

Mean Arterial Pressure (MAP) denotes the mean blood pressure of the artery.

Systolic Blood Pressure (SBP) denotes the blood pressure when the heart contracts.

Diastolic Blood Pressure (DBP) denotes the blood pressure when the heart is at rest.

Arterial Stiffness (AS) denotes the stiffness of the arterial walls.

BTLE or BLE stands for Bluetooth low energy that represents a feature of Bluetooth 4.2 wireless radio technology.

LCD stands for a liquid crystal display, and is a video display that uses the light modulating properties of liquid crystals.

USB stands for a Universal Serial Bus, and is an industry standard for cables, connectors, and communications protocols between computers and electronic devices.

ADC stands for an Analog-to-Digital Converter, and is a component that uses sampling to convert an analog quantity to a discrete time representation in digital form, i.e. it converts an analog signal into a digital signal.

App stands for application software, that is, computer software designed to help a user perform a specific task or tasks.

In one embodiment of the present disclosure, this wrist type apparatus is such as a wrist type blood pressure device that can be used to continuously monitor cardiovascular health. The cardiovascular health is monitored while using an electrocardiography (ECG) source synchronized to a piezoelectric pulse source. It is characterized in that the approach is without requiring invasive techniques or ongoing, large-scale external scanning procedures. The wrist type apparatus includes an electrode-based module acting as an ECG signal source with at least three ECG electrodes, wherein each ECG electrode connects to a corresponding contact window, contacting the skin for generating a first set of data by measuring a target heartbeat. The wrist type apparatus includes a piezoelectric sensor module which acts as a piezoelectric pulse signal source that generates a second set of data by measuring the pulse wave produced by the target heartbeat corresponding to the first set of data, wherein the piezoelectric sensor module comprises at least one piezoelectric pulse sensor connecting with a corresponding contact window that is used to contact skin. Incidentally, the “at least three ECG electrodes” means that it could be three or more than three ECG electrodes, and the “at least one piezoelectric pulse sensor” means that it could be one, two, three or more piezoelectric pulse sensors. The wrist type apparatus includes a processor, e.g., a microcontroller or microprocessor, which is configured to receive and process the first set of data and the second set of data. The processor receives the first and second sets of data from which the time differential of the heartbeat pulmonary pressure wave can be calculated. The continuous data related to cardiovascular health markers such as arterial stiffness can be determined. Selectively, the wrist type apparatus includes a display unit, e.g., LCD display, to show the information, especially at least one cardiovascular health value.

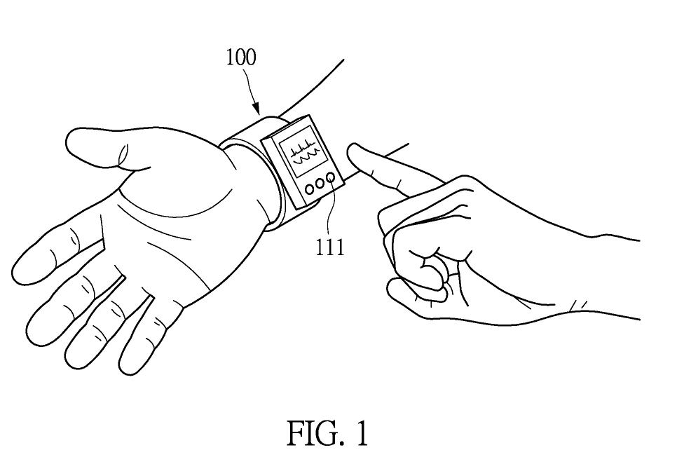

Reference is made to FIG. 1 illustrating a circumstance that a wrist type apparatus (100) worn on a user's right hand to measure user's cardiovascular health value. The wrist type apparatus (100) is such as a wrist type blood pressure device that includes at least three ECG electrodes and at least one piezoelectric pulse sensor.

To measure the cardiovascular health value, the user needs to wear the wrist type apparatus (100) on the user's hand, especially on the user's wrist, and use another hand to contact an ECG electrode (111) disposed on a side of a display. The wrist type apparatus (100) is capable of measuring ECG signals using its electrode-based module. The electrode-based module includes at least three ECG electrodes connecting with corresponding contact windows which contact the skin of the user for generating a first set of data by measuring a target heartbeat (not shown). The wrist type apparatus (100) is also capable of measuring a pulse wave produced by the target heartbeat using its piezoelectric sensor module. This piezoelectric sensor module comprising at least one piezoelectric pulse sensor connecting with a corresponding contact window which contacts the skin of the user is used to generate a second set of data by measuring the pulse wave corresponding to the first set of data.

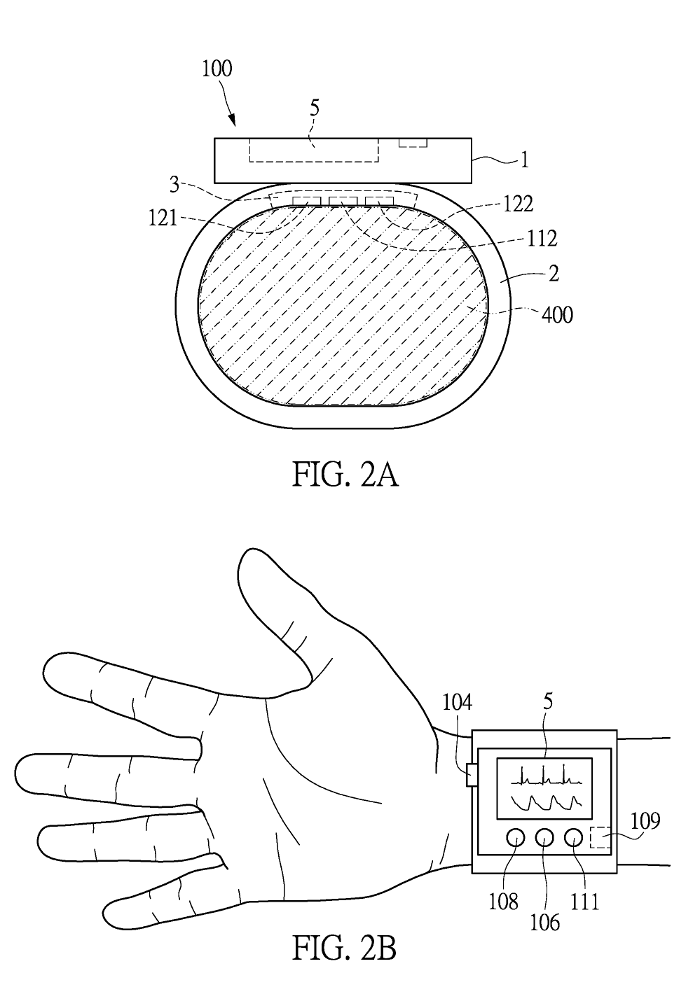

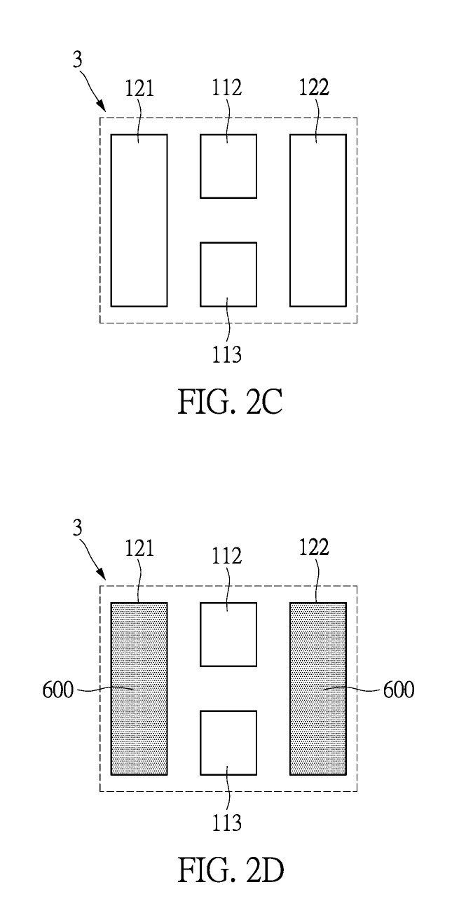

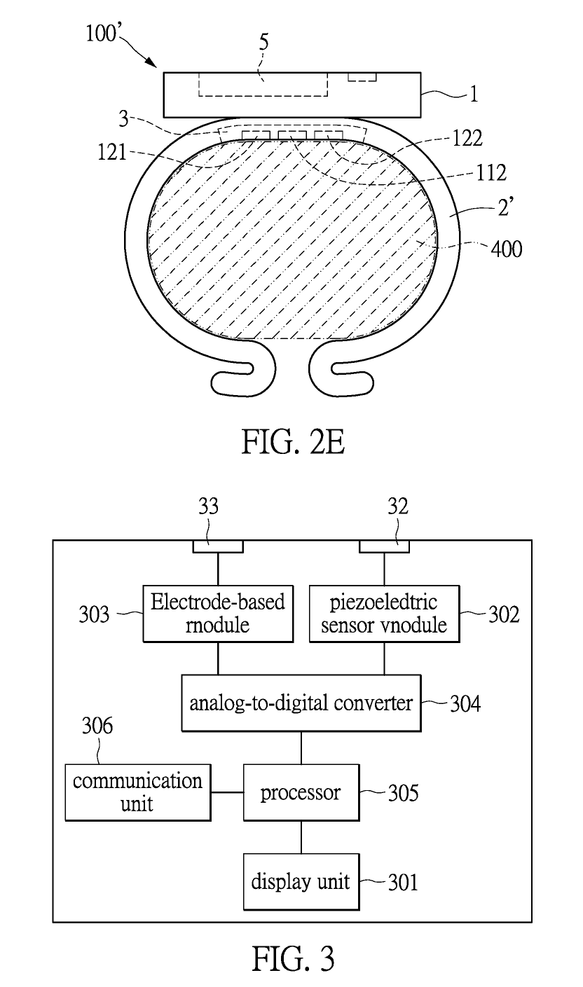

According to one of the embodiments of the present disclosure, the wrist type apparatus mentioned in FIG. 1 can be implemented by the schematic diagram described in FIG. 2A, FIG. 2B, FIG. 2C, FIG. 2D and FIG. 2E.

FIG. 2A shows a schematic diagram exemplarily depicting the cross section of the wrist type apparatus (100). On the cross section, the wrist type apparatus (100) worn on a user's wrist (400) comprises 2 parts, the first part is a main portion (1) and the second part is a cuff portion (2). The main portion (1) is electrically connected with the cuff portion (2). In this embodiment, a shape of the cuff portion (2) is an elastic wristband. A user's hand could wear this wrist type apparatus in a direct through way, and the wrist type apparatus would encircle the user's hand accordingly and tightly.

Specifically, the ECG electrode (111) is disposed on a same side of a display (5) in the main portion (2). Two ECG electrodes (112, 113) (113 isn't shown in this diagram), and two piezoelectric pulse sensors (121, 122) are disposed on a skin side, especially the top side, of the cuff portion (2). The skin side is an area which is in contact with the user's body. Therefore, the top side of the cuff portion (2) in this embodiment will form a special area so called a sensor array (3). The sensor array (3) in the present disclosure could be disposed in a special arrangement.

For example, an electrode-based module and a piezoelectric sensor module are included in the wrist type apparatus (100), and the electrode-based module comprising three ECG electrodes wherein each ECG electrode connects to a corresponding contact window, and the piezoelectric sensor module comprising two piezoelectric pulse sensors (121, 122) wherein each piezoelectric pulse sensor connects to a corresponding contact window, the contact windows being disposed over a surface of the wrist type apparatus, are provided. In this example, it is noted that the ECG signal is obtained through the corresponding contact windows of the at least three ECG electrodes that contact the skin of the user's body. The two ECG electrodes (112, 113) disposed on a top side of the cuff portion (2) contact the skin of the user's right wrist, and the one ECG electrode (111) disposed on a side of the display unit is touched by the user's left hand. The piezoelectric pulse signal is obtained through the one of two corresponding contact windows of the piezoelectric pulse sensors (121, 122) that contact the skin of the user's right wrist. In the present disclosure, only one piezoelectric pulse signal of the two piezoelectric pulse sensors (121, 122) is actually needed, but, for the user's convenience, whether the user wears the wrist type apparatus frontward or backward, the wrist type apparatus can have a good piezoelectric pulse signal reception (much closer to the Ulnar artery) under both circumstances accordingly.

FIG. 2B shows a schematic diagram exemplarily depicting a front side of the wrist type apparatus (100).

It schematically shows an antenna (104) indicative of its wireless communication capability, e.g., a built-in wireless communication module in compliance with Bluetooth® technology. The wrist type apparatus (100) includes a start key (106) which can be used to trigger the function of starting measurement of the ECG signals and/or the pulse wave produced by the target heartbeat. Further, an LCD display may be provided for displaying the data produced by the measurement. The LCD display may also show any other information produced by the apparatus (100). A power-on key (108) may be provided for the user to press to turn on the device. An ECG electrode (also called “first ECG electrode”) (111) is disposed at the same side of the LCD display. This ECG electrode (111) is used to form a lead. The wrist type apparatus (100) may be connected to the outside power supply via any communication port such as a USB port (109). Via this USB port (109), the wrist type apparatus (100) can also communicate with an external device, for example, for data synchronization, firmware updating, or a network connection.

It is noted that ECG can be used to measure the rate and rhythm of heartbeat, and this is a measurement of the distance between two consecutive heartbeats. To measure the ECG signals, the user can wear the wrist type apparatus (100) in one hand, e.g., the right hand shown in FIG. 1, and the skin of the right hand can contact two of the ECG electrodes (112, 113) disposed on the top side of the cuff portion; and then to contact the first ECG electrode (111) by the left hand.

FIGS. 2C and 2D schematically show the arrangement of the sensor array (3) of the wrist type apparatus 100.

In FIG. 2C, the sensor array (3) is composed of a pair of ECG electrodes (also called “second ECG electrode” and “third ECG electrode”) (112, 113) and a pair of piezoelectric pulse sensors (also called “first piezoelectric pulse sensor” and “second piezoelectric pulse sensor”) (121, 122). In which, the second piezoelectric pulse sensor is aligned with the first piezoelectric sensor, and the second and the third ECG electrodes are placed in between the first and the second piezoelectric pulse sensors.

In another embodiment, the piezoelectric sensor module includes a pair of a first piezoelectric pulse sensor and a second piezoelectric pulse sensor that are disposed at the skin side of the cuff portion in parallel.

In FIG. 2D, the schematic diagram depicts that the piezoelectric pulse sensors (121, 122) could be encapsulated with a layer of room temperature vulcanization (RTV) (600) for a better signal receiving effectiveness. Incidentally, the RTV silicone rubber compounds are clear liquids which cure at room temperature to high strength silicone rubber with the addition of curing agents. These two-component products are supplied with curing agent in matched kits which are designed for use at a convenient 10:1 ratio by weight.

FIG. 2E schematically shows another embodiment about a shape of a wrist type apparatus (100′). In this embodiment, the wrist type apparatus (100) is composed of a main portion (1) and a cuff portion (2′). The cuff portion (2′) is in the shape of a “C” such that a user could wear this wrist type apparatus in a quick clamping way. Except for the shape of the cuff portion (2′), the wrist type apparatus (100′) is the same as the wrist type (100) apparatus of the present disclosure.

Reference is next made to FIG. 3 depicting a block diagram of the wrist type apparatus. The block diagram shows several major hardware or software-implemented components of the wrist type apparatus (100) for embodying the functions of measurement of cardiovascular health according to one embodiment of the present disclosure.

The internal hardware components of the wrist type apparatus (100) are such as an electrode-based module (303), a piezoelectric sensor module (302), an analog-to-digital converter (304), a processor (305), and a display unit (301). Further, the wrist type apparatus (100) may also include a communication unit (306).

In one aspect of the present disclosure, the processor (305) is such as a microprocessor or a microcontroller adapted in the apparatus (100) for processing the internal operations. The processor (305) is electrically connected with the electrode-based module (303) and the piezoelectric sensor module (302). The processor (305) is essentially in communication with both the electrode-based module (303) and the piezoelectric sensor module (302) for receiving the first set of data and the second set of data and accordingly calculating at least one cardiovascular health value. In one embodiment, the analog-to-digital converter (304) may be used in the circuitry for converting the analog signals obtained via the interfaces (32, 33) (also called “contact window”) to digital signals for the processor (305).

Moreover, the electrode-based module (303) includes at least three ECG electrodes schematically represented by a first interface (33), e.g., corresponding contact windows of the at least three ECG electrodes (referred to FIG. 2C, 112, 113), which contact the skin of the human body for generating a first set of data by measuring a target heartbeat. The piezoelectric sensor module (302) may also have a second interface (32), e.g., corresponding contact windows of the at least one piezoelectric pulse sensor (referred to FIG. 2C, 121, 122), which contact the skin of the human body for generating a second set of data by measuring a pulse wave produced by the target heartbeat corresponding to the first set of data.

In one aspect of the present disclosure, the electrode-based module (303) is an electrocardiographic source that includes at least three ECG electrodes wherein each ECG electrode has its corresponding contact window represented by the first interface (33) disposed over a surface of the wrist type apparatus (100). In operation of the wrist type apparatus (100), the wrist type apparatus (100) receives analog ECG signals from the electrode-based module (303) via the corresponding contact windows (33) of the at least three ECG electrodes contacted with the skin.

The piezoelectric sensor module (302) may be a piezoelectric pulse source that has the external second interface (32). To gain the pulse wave of the heartbeat, the wrist type apparatus (100) receives the pulse wave from the piezoelectric sensor module (302) via the interface (32).

The analog-to-digital converter (304) is electrically connected with the electrode-based module (303) and the piezoelectric sensor module (302) and is used to convert the analog signals into digital signals that are conveyed to the processor (305).

In one further aspect of the present disclosure, the wrist type apparatus (100) includes a communication unit (306). The communication unit (306) may be a wireless communication component for implementing the wireless connection with an external device, or a cloud server for collecting data from the wrist type apparatus (100).

Furthermore, a pulse transit time (PTT) is one of the cardiovascular health markers for assessing cardiovascular health. To obtain the pulse transit time, the wrist type apparatus (100) first receives the ECG signals and the piezoelectric pulse signals from the target heartbeat, and these two sets of data are synchronized by the processor (305) and used to calculate the pulse transit time. The related cardiovascular markers can be therefore derived.

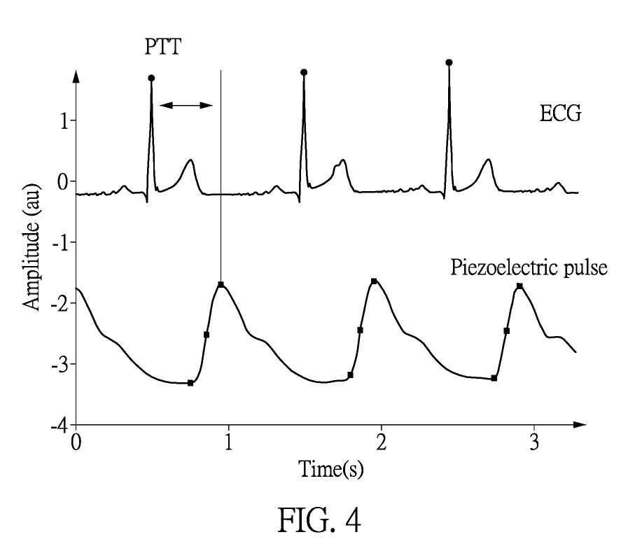

Reference is made to FIG. 4, showing the signal records about the ECG signals and the piezoelectric pulse signals in the target heartbeat cycle.

In the diagram, the peak amplitude of the target heartbeat is detected by the ECG The peak amplitude of the corresponding pulse wave in the blood vessels is detected by the piezoelectric pulse sensor. The difference in time between these peak amplitudes can be recognized as the pulse transit time (PTT). By this method, other cardiovascular health markers can also be calculated. For example, the pulse wave velocity (PWV) is the distance traveling by the pulse divided by PTT (PWV=distance/PTT). It is noted that it is closely approximated by the distance between the ECG and piezoelectric pulse sources, or the measured length of the artery from the target heart to the end of the arm, e.g., the fingers, as exemplarily shown in FIG. 1. The arterial stiffness can also be derived from PWV through the Moens-Korteweg equation.

Alternatively, other specific endpoints in the pulse waveform as detected by the PPG may be used to determine the PTT. Possibilities include, but are not limited to, the peak, the midpoint, the foot, the point of maximal slope, and the virtual basepoint. It is noted that the virtual base point corresponds to the intersection point between the tangent to the pulse wave at the point of maximal slope and the horizontal line going through the point having the absolute minimum signal. Different endpoints are suggested to have different advantages in measuring and using the PTT value. For example, using the virtual basepoint has been suggested to give a better virtual noise and artifact robustness. Using the point of maximal slope has been suggested to be strongly related to SBP (systolic blood pressure).

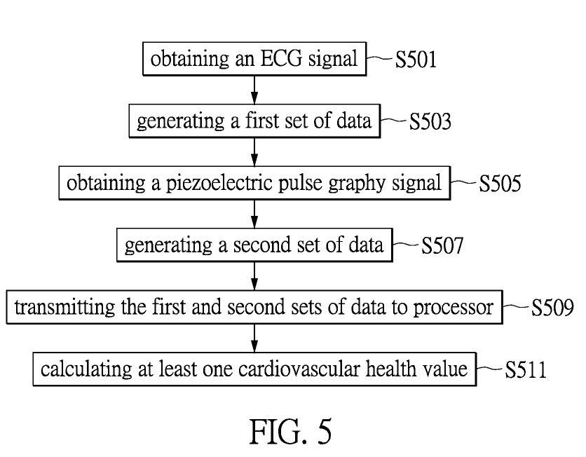

In one further embodiment of the present disclosure, a method for measurement of cardiovascular health by monitoring at least one cardiovascular health marker over time is disclosed as described in the flow chart shown in FIG. 5.

To obtain the pulse transit time, the embodiments in the disclosure are applicable to the user's wrist type apparatus that is used to obtain the ECG signals and the piezoelectric pulse signals. The two sets of data are synchronized by the processor of the apparatus and used to calculate pulse transit time and related cardiovascular health markers such as arterial stiffness. In this manner, the method becomes able to continuously derive measurements relating to arterial stiffness and associated cardiovascular markers with this synchronized set of ECG and Piezoelectric Pulse sources. The measurements of data stream sequences are collected from subsequent data ranges and can then be compared to verify cardiovascular trending markers corresponding to a specific individual's rate of arterial stiffness and circulatory degeneration, effectively providing a personalized biometric trending signature. The approach can render preventive measures that can be potentially applied before a health crisis occurs.

In the embodiment of the method shown in FIG. 5, the arterial stiffness of the user who wears the wrist type apparatus is monitored. The systems including the wrist type apparatus of the present disclosure can be configured to measure, calculate, or estimate one or more cardiovascular health markers over time, including, but not limited to, arterial stiffness, blood pressure, heart rate, pulse transit time, and pulse wave velocity. Further, at least two of arterial stiffness, BP, HR, PTT, and PWV can be monitored over time. Still further, at least three of arterial stiffness, BP, HR, PTT, and PWV can be monitored over time. In another embodiment, all of arterial stiffness, BP, HR, PTT, and PWV are monitored over time.

To obtain the data generated by the wrist type apparatus, a wireless communication technology is used in the wrist type apparatus. For example, Bluetooth® technology may be used to process the communication. More, the wrist type apparatus may conduct the communication via any data port such as USB. The USB port is one of the schemes that are compatible for computer data transferring and also for battery recharging.

The combination of the signal measurement and data interpolation derived from protracted sequences of continuous monitoring output according to the present disclosure increase the accuracy of the measurement. It allows for determination of arterial health trending markers over time. Data derived from a continuous measurement process provides more accurate analysis of cardiovascular health indicators beyond intermittent measurements such as BP, enables derivation of individual biometric trending that can account for anomalous BP, PTT, or PWV values due to moments of stress and other health and environmental triggers.

Continuous, unobtrusive, portable and wearable monitoring approaches disclosed in the present disclosure can be rendered as strong applications for telemedicine purposes. Without limitation, these monitoring methods are used to remotely validate rehabilitation compliance or fitness goals. It can be used in the doctor's office, in the hospital, at home, and during the individual's daily activities. It has potential for use not only in the medical and fitness fields, but also for monitoring purposes in health insurance, policing, athletics, and military defense. It can be used to remotely store, e.g., via cloud server, or selectively display cardiovascular health data about one or more individuals over a period of time, including during healthy or ill stages, and in determining health marker changes due to disease or aging. Those of ordinary skill in the art could identify a range of practical uses for the present embodiments.

With the sustained synchronous and continuous nature of the present embodiments, there is potential for more accurate PPG measurement of arterial performance efficiency, as opposed to the arterial measurement of the prior art. The wrist type apparatus, e.g., the wrist type blood pressure devices, of the present embodiments offers greater usage convenience and portability over the conventional technologies for care of cardiovascular health.

A major advantage of the present disclosure is the ability to continuously monitor cardiovascular health. An even greater advantage arises with the present disclosure when the wrist type blood pressure device comprises both ECG signal source and piezoelectric pulse source, in which all of the components required are in the device to continuously monitor cardiovascular health conveniently and unobtrusively, and those components are disposed in a single, compact, wrist type device, at a single point on the body.

Links have been suggested throughout the prior art between the values of PTT, PWV, changes in BP, and arterial stiffness. It is generally accepted that both PTT and PWV can be regarded as indices of arterial stiffness, and that both can also be employed as estimators of BP. HR is easily monitored with either the ECG or Piezoelectric Pulse sources by measuring beats per unit time (typically referred-to as beats per minute, or bpm). The present disclosure may be configured to calculate or estimate any or all of arterial stiffness, BP, HR, PTT, and PWV through continuous monitoring.

In FIG. 5, a flow chart is shown to disclose the method for measurement of cardiovascular health by monitoring at least one cardiovascular health marker over time in one embodiment of the present disclosure. Using the wrist type apparatus (100) of FIG. 2A, essentially including an electrode-based module, a piezoelectric sensor module, and a processor, in step S501, an ECG signal for a target heartbeat can be obtained using the electrode-based module of the wrist type apparatus. This electrode-based module essentially includes at least three electrodes which are used to contact the skin of the human body, and therefore generates a first set of data by measuring the target heartbeat, such as in step S503. Further, in step S505, a piezoelectric pulse graph signal for a pulse wave from the target heartbeat is obtained using the piezoelectric sensor module of the wrist type apparatus. The piezoelectric sensor module then generates a second set of data by measuring a pulse wave produced by the target heartbeat corresponding to the first set of data, as shown in step S507.

After that, in step S509, after the first set of data and the second set of data are generated, the data are transmitted to the processor of the wrist type apparatus through the internal electronic lines. The processor, electrically connected with the electrode-based module and the piezoelectric sensor module, is in communication with both the electrode-based module and the piezoelectric sensor module for receiving the first set of data and the second set of data. In the next step S511, by the processor, at least one cardiovascular health value can be calculated in accordance with the at least one cardiovascular health marker using the first and second sets of data.

Furthermore, the system provides a cloud server in one aspect of the present disclosure, and the cloud server is connected with the one or more wrist type apparatuses over a network. The cloud server is used to collect the cardiovascular health values from a plurality of wrist type apparatuses at different locations.

The cardiovascular health marker is selected from arterial stiffness, blood pressure, heart rate, pulse transit time, and pulse wave velocity. For example, in the step for calculating the at least one cardiovascular health marker, the pulse transit time is one of cardiovascular markers and can be calculated at this phase, and can also be continuously monitored. Further, the system can be used to continuously measure arterial stiffness and related cardiovascular markers of the user for the purpose of long-term health monitoring. In an exemplary embodiment, the wrist type apparatus is the major tool for implementing long-term monitoring for at least one cardiovascular health marker over time after the at least one cardiovascular health value is calculated.

Accordingly, through prolonged and uninterrupted data measurement, the wrist type apparatus can be used to collect data across multiple independent sequential time ranges and compare them with each other so as to determine a rate of change in accordance with every cardiovascular health marker. Through the system, the rate of change in accordance with every cardiovascular health marker for every wrist type apparatus can be transmitted to the cloud server. The cloud server then allows every wrist type apparatus to synchronize the data there-between.

Many prior arts focus on trying to obtain a fixed BP value for an individual to determine their cardiovascular health. Rather than trying to calculate the fixed BP value, one of the objectives of the disclosure is to determine the degeneration of arterial elasticity over time. In one embodiment, once a pulse transit time is calculated, the pulse wave velocity is derived. A suitable formula for linking pulse wave velocity and arterial stiffness is the Moens-Korteweg equation:

PWV=√[(E_{inc}·h)/(2rρ)]

The Moens-Korteweg equation states that PWV is proportional to the square root of the incremental elastic modulus, (E_{inc}), of the vessel wall given constant ratio of wall thickness, h, to vessel radius, r, and blood density, ρ, assuming that the artery wall is isotropic and experiences isovolumetric change with pulse pressure.

Because of the constant ratio of wall thickness, h, to vessel radius, r, and blood density, ρ, PWV can be used as a direct correlation to arterial stiffness. With monitoring over time, changes in an individual's PWV can be directly linked to changes in arterial stiffness.

Alternately phrased, the Moens-Korteweg equation can be stated as follows:

PWV=√[(tE)/(ρd)]

where t is vessel wall thickness, ρ is blood density, d is the interior diameter of the vessel. As previously stated, PWV also equals the length of the vessel (L) traveled by the pulse divided by the PTT (T):

PWV=L/T

The elastic modulus, E, is indicated as:

E=E₀e^{αP}

wherein E₀ is the modulus at zero pressure, α is dependent on the vessel, and P is the blood pressure. Making the appropriate combinations and substitutions into the Moens-Korteweg equation yields:

L/T=√[(tE₀e^{αP})/(ρd)]

which leads to:

P=(1/α[In(L²ρd/tE₀)−(2InT)]

If changes to wall thickness t and diameter of the vessel d with respect to changes to blood pressure P are negligible, and the change in the modulus E₀ is slow enough, the change in blood pressure can be linearly related to the change in PTT as follows:

ΔP=(−2/αT)ΔT

Similarly, through the Bramwell-Hill equation:

PWV=√[(ΔP·V)/(ρ·ΔV)]

where V is blood volume, ΔV is change in blood volume, ΔP is change in blood pressure, and ρ is blood density.

As found in the prior art through both the Moens-Korteweg and Bramwell-Hill equations, both PWV and PTT have been established to have approximate linear relationships to systolic and diastolic or mean blood pressure (P), according to the following equations:

P=(1/α)(PWV−b), and

P=(1/n)(m−PTT)

where a, b, m, and n are user or patient-specific constants.

In another embodiment of the present disclosure, the value of the BP could be calibrated to provide a more accurate value. According to some studies, an accurate noninvasive estimation of mean arterial pressure (estimated MAP) is of great importance in the evaluation of circulatory function and prognosis of some cardiovascular diseases. The present disclosure provides a BP calibration method by the estimated MAP and a calculated MAP (defined later). Based on the pulse transit time (PTT), an equation of the estimated MAP can be presented as a following equation:

estimated MAP=A*P77+B

In which, the value of PTT computed as the time interval between the electrocardiogram (ECG) R-peaks and the peak of pulses is mathematically modeled by the MCU mentioned above, the “A” is a customized parameter for the calibration, and the “B” is another customized parameter for the calibration. The calculated MAP can be presented as a following equation:

calculated MAP=0.33*SBP+0.66*DBP

, wherein the SBP is systolic blood pressure and the DBP is the diastolic blood pressure. This BP calibration method uses the difference of the value of calculated MAP and the value of estimated MAP to calibrate the BP.

The above-mentioned descriptions represent merely the exemplary embodiment of the present disclosure, without any intention to limit the scope of the present disclosure thereto. Various equivalent changes, alterations or modifications based on the claims of the present disclosure are all consequently viewed as being embraced by the scope of the present disclosure.

What is claimed is:

1. A wrist type apparatus for measurement of cardiovascular health which comprises a main portion connected with a cuff portion, comprising:
a display unit disposed on the main portion;
an electrode-based module, used to generate a first set of data by measuring a target heartbeat, comprising at least three electrocardiography (ECG) electrodes, wherein each ECG electrode connects to a corresponding contact window that is used to contact skin;
a piezoelectric sensor module, used to generate a second set of data by measuring a pulse wave produced by the target heartbeat corresponding to the first set of data, comprising at least one piezoelectric pulse sensor, wherein each piezoelectric pulse sensor connects to a corresponding contact window that is used to contact skin; and
a processor, electrically connected with the display unit, electrode-based module and the piezoelectric sensor module, in communication with both the electrode-based module and the piezoelectric sensor module for receiving the first set of data and the second set of data and accordingly calculating at least one cardiovascular health value being shown on the display unit;
wherein, the at least three ECG electrodes has a first ECG electrode, a second ECG electrode and a third ECG electrode, in which the first ECG electrode is disposed on a same side of the display unit; the second ECG electrode, the third ECG electrode and one piezoelectric pulse sensor are disposed on a skin side of the cuff portion.

2. The wrist type apparatus as recited in claim 1, wherein the wrist type apparatus is a wrist type blood pressure device which calibrates a value of blood pressure by following steps:
calculating an estimation of mean arterial pressure (estimated MAP), wherein the estimated MAP is equal to a parameter A multiplying by a value of pulse transit time (PTT) add a parameter B;
calculating a calculation of mean arterial pressure (calculated MAP), wherein a value of the calculated MAP is equal to a value of systolic blood pressure (SBP) multiplying by 0.33 add a value of diastolic blood pressure (DBP) multiplying by 0.66;
calibrating the blood pressure by the difference of the value of the calculated MAP and the value of the estimated MAP.

3. The wrist type apparatus as recited in claim 1, wherein the piezoelectric sensor module forms a piezoelectric pulse source and the electrode-based module forms an electrocardiographic source.

4. The wrist type apparatus as recited in claim 3, wherein the electrocardiographic source comprises the corresponding contact window for each ECG electrode, and the piezoelectric pulse source comprises the corresponding contact window for each piezoelectric pulse sensor.

5. The wrist type apparatus as recited in claim 3, further comprising an analog-to-digital converter used to convert an analog signal obtained from the contact windows of the electrode-based module or the contact window of the piezoelectric sensor module into a digital signal that is conveyed to the processor.

6. The wrist type apparatus as recited in claim 1, wherein the piezoelectric sensor module includes a pair of a first piezoelectric pulse sensor and a second piezoelectric pulse sensor that are disposed at the skin side of the cuff portion in parallel.

7. The wrist type apparatus as recited in claim 1, the corresponding contact window of the piezoelectric pulse sensor is encapsulated by a room temperature vulcanization (RTV) silicone rubber.

8. A method for measurement of cardiovascular health by monitoring at least one cardiovascular health marker over time, adapted to the wrist type apparatus of claim 1, comprising:
obtaining an ECG signal for the target heartbeat so as to generate the first set of data when a first area of a user's body is contacted with the corresponding contact windows of the second ECG electrode and the third ECG electrode, and a second area of the user's body touches the corresponding contact window of the first ECG electrode;
obtaining a piezoelectric pulse signal for a pulse wave from the target heartbeat so as to generate the second set of data when the first area of the user's body is contacted with the corresponding contact window of the at least one piezoelectric pulse sensor;
transmitting the first set of data and the second set of data to the processor of the wrist type apparatus; and
by the processor, calculating the at least one cardiovascular health value in accordance with the at least one cardiovascular health marker using the first and second sets of data.

9. The method as recited in claim 8, wherein the wrist type apparatus is used to continuously monitor the at least one cardiovascular health marker over time after the at least one cardiovascular health value is calculated.

10. The method as recited in claim 9, wherein the cardiovascular health marker is selected from arterial stiffness, blood pressure, heart rate, pulse transit time, and pulse wave velocity.

11. The method as recited in claim 10, wherein a value of the blood pressure calibrated by following steps:
calculating an estimation of mean arterial pressure (estimated MAP), wherein the estimated MAP is equal to a parameter A multiplying by a value of pulse transit time (PTT) add a parameter B;
calculating a calculation of mean arterial pressure (calculated MAP), wherein a value of the calculated MAP is equal to a value of systolic blood pressure (SBP) multiplying by 0.33 add a value of diastolic blood pressure (DBP) multiplying by 0.66;
calibrating the value of the blood pressure by the difference of the value of the calculated MAP and the value of the estimated MAP.

12. The method as recited in claim 10, wherein, through a prolonged uninterrupted data measurement, further comprising collecting across multiple independent sequential time ranges and comparing with each other so as to determine a rate of change in accordance with every cardiovascular health marker.

13. The method as recited in claim 8, wherein the corresponding contact window of the at least one piezoelectric pulse sensor is encapsulated with a room temperature vulcanization (RTV) silicone rubber.

14. The method as recited in claim 8, wherein the piezoelectric sensor module includes a pair of a first piezoelectric pulse sensor and a second piezoelectric pulse sensor that are disposed at the skin side of the cuff portion in parallel.

15. A system for measurement of cardiovascular health, comprising:
one or more wrist type apparatuses which comprise a main portion connected with a cuff portion, each comprising:
a display unit disposed on the main portion;
an electrode-based module, used to generate a first set of data by measuring a target heartbeat, comprising at least three electrocardiography (ECG) electrodes, wherein each ECG electrode connects to a corresponding contact window that is used to contact skin;
a piezoelectric sensor module, used to generate a second set of data by measuring a pulse wave produced by the target heartbeat corresponding to the first set of data, comprising at least one piezoelectric pulse sensor, wherein each piezoelectric pulse sensor connects to a corresponding contact window that is used to contact skin; and
a processor, electrically connected with the display unit, electrode-based module and the piezoelectric sensor module, in communication with both the electrode-based module and the piezoelectric sensor module for receiving the first set of data and the second set of data and accordingly calculating at least one cardiovascular health value being shown on the display unit;
wherein, the at least three ECG electrodes has a first ECG electrode, a second ECG electrode and a third ECG electrode, in which the first ECG electrode is disposed on a same side of the display unit; the second ECG electrode, the third ECG electrode and one piezoelectric pulse sensor are disposed on a skin side of the cuff portion;
and
a cloud server, connected with the one or more wrist type apparatuses over a network, used to collect the cardiovascular health value from every wrist type apparatus.

16. The system as recited in claim 15, wherein every wrist type apparatus continuously monitors the at least one cardiovascular health marker over time for continuously calculating the at least one cardiovascular health value.

17. The system as recited in claim 16, wherein the cardiovascular health marker is selected from arterial stiffness, blood pressure, heart rate, pulse transit time, and pulse wave velocity.

18. The system as recited in claim 17, wherein a value of the blood pressure calibrated by following steps:
calculating an estimation of mean arterial pressure (estimated MAP), wherein the estimated MAP is equal to a parameter A multiplying by a value of pulse transit time (PTT) add a parameter B;
calculating a calculation of mean arterial pressure (calculated MAP), wherein a value of the calculated MAP is equal to a value of systolic blood pressure (SBP) multiplying by 0.33 add a value of diastolic blood pressure (DBP) multiplying by 0.66;
calibrating the value of the blood pressure by the difference of the value of the calculated MAP and the value of the estimated MAP.

19. The system as recited in claim 15, wherein, through a prolonged uninterrupted data measurement, the wrist type apparatus collects across multiple independent sequential time ranges and compares with each other so as to determine a rate of change in accordance with every cardiovascular health marker.

20. The system as recited in claim 19, wherein the rate of change in accordance with every cardiovascular health marker for every wrist type apparatus is transmitted to the cloud server.

21. The system as recited in claim 20, wherein the cloud server allows every wrist type apparatus to synchronize the data there-between.

22. The system as recited in claim 15, wherein the corresponding contact window of the piezoelectric pulse sensor is encapsulated by a room temperature vulcanization (RTV) silicone rubber.
